(19) United States
(12) Patent Application Publication
(10) Pub. No.: US 2020/0327379 A1
(43) Pub. Date: Oct. 15, 2020

(54) FASTESTIMATOR HEALTHCARE AI FRAMEWORK

(71) Applicant: GE Precision Healthcare LLC, Wauwatosa, WI (US)

(72) Inventors: Xiaomeng Dong, San Ramon, CA (US); Aritra Chowdhury, San Ramon, CA (US); Junpyo Hong, San Ramon, CA (US); Hsi-Ming Chang, San Ramon, CA (US); Gopal B. Avinash, San Ramon, CA (US); Venkata Ratnam Saripalli, San Ramon, CA (US); Karley Yoder, Oakland, CA (US); Michael Potter, Danville, CA (US)

(21) Appl. No.: 16/699,567

(22) Filed: Nov. 30, 2019

Related U.S. Application Data
(60) Provisional application No. 62/831,485, filed on Apr. 9, 2019.

(51) Int. Cl. G06K 9/62; G06N 3/08; G06N 3/04

(52) U.S. Cl. CPC G06K 9/6263; G06N 3/0454; G06N 3/08; G06K 9/6256

(57) ABSTRACT

An artificial intelligence platform and associated methods of training and use are disclosed. An example apparatus includes a data pipeline to: preprocess data using one or more preprocessing operations applied to features associated with the data; and enable debugging to visualize the preprocessed data. The example apparatus includes a network to: instantiate one or more differentiable operations in a training configuration to train an artificial intelligence model; capture feedback including optimization and loss information to adjust the training configuration; and store one or more metrics to evaluate performance of the artificial intelligence model. The example apparatus includes an estimator to: store the training configuration for the artificial intelligence model; configure the pipeline and the network based on the training configuration; iteratively link the pipeline and the network based on the training configuration; and initiate training of the artificial intelligence model using the linked pipeline and network.

### CROSS-REFERENCE TO RELATED APPLICATIONS

This patent arises from U.S. Provisional Patent Application Ser. No. 62/831,485, which was filed on Apr. 9, 2019. U.S. Provisional Patent Application Ser. No. 62/831,485 is hereby incorporated herein by reference in its entirety. Priority to U.S. Provisional Patent Application Ser. No. 62/831,485 is hereby claimed.

### FIELD OF THE DISCLOSURE

This disclosure relates generally to a healthcare artificial intelligence framework and, more particularly, to a healthcare artificial intelligence framework and methods of use.

### BACKGROUND

The statements in this section merely provide background information related to the disclosure and may not constitute prior art.

Healthcare environments, such as hospitals or clinics, include information systems, such as hospital information systems (HIS), radiology information systems (RIS), clinical information systems (CIS), and cardiovascular information systems (CVIS), and storage systems, such as picture archiving and communication systems (PACS), library information systems (LIS), and electronic medical records (EMR). Information stored can include patient medication orders, medical histories, imaging data, test results, diagnosis information, management information, and/or scheduling information, for example. A wealth of information is available, but the information can be siloed in various separate systems requiring separate access, search, and retrieval. Correlations between healthcare data remain elusive due to technological limitations on the associated systems.

Artificial intelligence systems can help to leverage the vast body of healthcare data and help to provide correlations and predictions involving the data. However, developing, training, testing, and deploying artificial intelligence model constructs remains a difficult, demanding, and highly specialized endeavors. Platforms or frameworks developed to aid in artificial model development have been lacking in functionality, connectivity, reliability, usability, and expressiveness to implement a wide range of tasks. As such, there is an unmet need for improved artificial intelligence prototyping and productization platforms.

### BRIEF DESCRIPTION

Systems, apparatus, instructions, and methods to implement, test, and execute networks of models to complete artificial intelligence tasks are disclosed.

Certain examples provide an artificial intelligence modularization apparatus. The example apparatus includes a data pipeline to: preprocess data using one or more preprocessing operations applied to features associated with the data; and enable debugging to visualize the preprocessed data. The example apparatus includes a network to: instantiate one or more differentiable operations in a training configuration to train an artificial intelligence model; capture feedback including optimization and loss information to adjust the training configuration; and store one or more metrics to evaluate performance of the artificial intelligence model. The example apparatus includes an estimator to: store the training configuration for the artificial intelligence model; configure the pipeline and the network based on the training configuration; iteratively link the pipeline and the network based on the training configuration; and initiate training of the artificial intelligence model using the linked pipeline and network.

Certain examples provide at least one computer readable storage medium including instructions that, when executed, cause at least one processor to implement at least: a data pipeline, a network, and an estimator. The example data pipeline is to: preprocess data using one or more preprocessing operations applied to features associated with the data; and enable debugging to visualize the preprocessed data. The example network is to: instantiate one or more differentiable operations in a training configuration to train an artificial intelligence model; capture feedback including optimization and loss information to adjust the training configuration; and store one or more metrics to evaluate performance of the artificial intelligence model. The example estimator is to: store the training configuration for the artificial intelligence model; configure the pipeline and the network based on the training configuration; iteratively link the pipeline and the network based on the training configuration; and initiate training of the artificial intelligence model using the linked pipeline and network.

Certain examples provide a computer-implemented method including preprocessing, with a data pipeline, data using one or more preprocessing operations applied to features associated with the data. The example method includes enabling, with the data pipeline, debugging to visualize the preprocessed data. The example method includes instantiating, with a network one or more differentiable operations in a training configuration to train an artificial intelligence model. The example method includes capturing, with the network, feedback including optimization and loss information to adjust the training configuration. The example method includes storing, with the network one or more metrics to evaluate performance of the artificial intelligence model. The example method includes storing, with an estimator, the training configuration for the artificial intelligence model. The example method includes configuring, with the estimator, the pipeline and the network based on the training configuration. The example method includes iteratively linking, with the estimator, the pipeline and the network based on the training configuration. The example method includes initiating, with the estimator, training of the artificial intelligence model using the linked pipeline and network.

### BRIEF DESCRIPTION OF DRAWINGS

FIG. 1 illustrates an example comparison of artificial intelligence frameworks.

FIG. 2 illustrates an example comparison in supported functionality between FastEstimator and other artificial intelligence frameworks.

FIG. 3 provides an example training speed comparison among artificial intelligence frameworks.

FIG. 4 depicts user populations covered by a plurality of artificial intelligence frameworks.

FIG. 5 illustrates example components and associated workflow of a FastEstimator artificial intelligence framework.

FIG. 6 illustrates an example implementation of the modular design of the FastEstimator artificial intelligence framework.

FIG. 7 illustrates an example implementation of a data pipeline and its modular data pipeline preprocessing of the example of FIG. 6.

FIG. 8 depicts an example FastEstimator framework to facilitate artificial intelligence training workflows.

FIG. 9 shows how an example deep learning application can be expressed as a sequence of operators.

FIG. 10 shows example operator graphs and associated expressions.

FIG. 11 shows an example training loop including an epoch loop and a batch loop.

FIG. 12 shows an example operation in which a plurality of traces propagate data to increase reusability of results.

FIGS. 13-17 depict example operator expressions for various deep learning tasks to be implemented using the FastEstimator platform.

FIGS. 18-20 illustrate flow diagrams of example methods to dynamically generate artificial intelligence systems and associated tasks using the FastEstimator platform.

FIG. 21 is a block diagram of an example processor platform capable of executing instructions to implement the example systems and methods disclosed and described herein.

### DETAILED DESCRIPTION OF CERTAIN EXAMPLES

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe example implementations and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects, and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities, and/or steps, the phrase “at least one of A or B” is intended to refer to implementations including any of (1) at least one A, (2) at least one B, and (3) at least one A and at least one B.

In addition, it should be understood that references to “one embodiment” or “an embodiment” of the present disclosure are not intended to be interpreted as excluding the existence of additional embodiments that also incorporate the recited features.

While certain examples are described below in the context of medical or healthcare systems, other examples can be implemented outside the medical environment.

### I. Overview

Medical data can be obtained from imaging devices, sensors, laboratory tests, and/or other data sources. Alone or in combination, medical data can assist in diagnosing a patient, treating a patient, forming a profile for a patient population, influencing a clinical protocol, etc. However, to be useful, medical data must be organized properly for analysis and correlation beyond a human's ability to track and reason. Computers and associated software and data constructs can be implemented to transform disparate medical data into actionable results.

For example, imaging devices (e.g., gamma camera, positron emission tomography (PET) scanner, computed tomography (CT) scanner, X-Ray machine, magnetic resonance (MR) imaging machine, ultrasound scanner, etc.) generate medical images (e.g., native Digital Imaging and Communications in Medicine (DICOM) images) representative of the parts of the body (e.g., organs, tissues, etc.) to diagnose and/or treat diseases. Medical images may include volumetric data including voxels associated with the part of the body captured in the medical image. Medical image visualization software allows a clinician to segment, annotate, measure, and/or report functional or anatomical characteristics on various locations of a medical image. In some examples, a clinician may utilize the medical image visualization software to identify regions of interest with the medical image.

Acquisition, processing, analysis, and storage of medical image data play an important role in diagnosis and treatment of patients in a healthcare environment. A medical imaging workflow and devices involved in the workflow can be configured, monitored, and updated throughout operation of the medical imaging workflow and devices. Machine learning can be used to help configure, monitor, and update the medical imaging workflow and devices.

Machine learning techniques, whether deep learning networks or other experiential/observational learning system, can be used to locate an object in an image, understand speech and convert speech into text, and improve the relevance of search engine results, for example. Deep learning is a subset of machine learning that uses a set of algorithms to model high-level abstractions in data using a deep graph with multiple processing layers including linear and non-linear transformations. While many machine learning systems are seeded with initial features and/or network weights to be modified through learning and updating of the machine learning network, a deep learning network trains itself to identify “good” features for analysis. Using a multilayered architecture, machines employing deep learning techniques can process raw data better than machines using conventional machine learning techniques. Examining data for groups of highly correlated values or distinctive themes is facilitated using different layers of evaluation or abstraction.

Throughout the specification and claims, the following terms take the meanings explicitly associated herein, unless the context clearly dictates otherwise. The term “deep learning” is a machine learning technique that utilizes multiple data processing layers to recognize various structures in data sets and classify the data sets with high accuracy. A deep learning network (DLN), also referred to as a deep neural network (DNN), can be a training network (e.g., a training network model or device) that learns patterns based on a plurality of inputs and outputs. A deep learning network/deep neural network can be a deployed network (e.g., a deployed network model or device) that is generated from the training network and provides an output in response to an input.

The term “supervised learning” is a deep learning training method in which the machine is provided already classified data from human sources. The term “unsupervised learning” is a deep learning training method in which the machine is not given already classified data but makes the machine useful for abnormality detection. The term “semi-supervised learning” is a deep learning training method in which the machine is provided a small amount of classified data from human sources compared to a larger amount of unclassified data available to the machine.

The term “convolutional neural networks” or “CNNs” are biologically inspired networks of interconnected data used in deep learning for detection, segmentation, and recognition of pertinent objects and regions in datasets. CNNs evaluate raw data in the form of multiple arrays, breaking the data in a series of stages, examining the data for learned features.

The term “transfer learning” is a process of a machine storing the information used in properly or improperly solving one problem to solve another problem of the same or similar nature as the first. Transfer learning may also be known as “inductive learning”. Transfer learning can make use of data from previous tasks, for example.

The term “active learning” is a process of machine learning in which the machine selects a set of examples for which to receive training data, rather than passively receiving examples chosen by an external entity. For example, as a machine learns, the machine can be allowed to select examples that the machine determines will be most helpful for learning, rather than relying only an external human expert or external system to identify and provide examples.

The term “computer aided detection” or “computer aided diagnosis” refer to computers that analyze medical images for the purpose of suggesting a possible diagnosis.

Deep learning is a class of machine learning techniques employing representation learning methods that allows a machine to be given raw data and determine the representations needed for data classification. Deep learning ascertains structure in data sets using backpropagation algorithms which are used to alter internal parameters (e.g., node weights) of the deep learning machine. Deep learning machines can utilize a variety of multilayer architectures and algorithms. While machine learning, for example, involves an identification of features to be used in training the network, deep learning processes raw data to identify features of interest without the external identification.

Deep learning in a neural network environment includes numerous interconnected nodes referred to as neurons. Input neurons, activated from an outside source, activate other neurons based on connections to those other neurons which are governed by the machine parameters. A neural network behaves in a certain manner based on its own parameters. Learning refines the machine parameters, and, by extension, the connections between neurons in the network, such that the neural network behaves in a desired manner.

Deep learning that utilizes a convolutional neural network segments data using convolutional filters to locate and identify learned, observable features in the data. Each filter or layer of the CNN architecture transforms the input data to increase the selectivity and invariance of the data. This abstraction of the data allows the machine to focus on the features in the data it is attempting to classify and ignore irrelevant background information.

Deep learning operates on the understanding that many datasets include high level features which include low level features. While examining an image, for example, rather than looking for an object, it is more efficient to look for edges which form motifs which form parts, which form the object being sought. These hierarchies of features can be found in many different forms of data such as speech and text, etc.

Learned observable features include objects and quantifiable regularities learned by the machine during supervised learning. A machine provided with a large set of well classified data is better equipped to distinguish and extract the features pertinent to successful classification of new data.

A deep learning machine that utilizes transfer learning may properly connect data features to certain classifications affirmed by a human expert. Conversely, the same machine can, when informed of an incorrect classification by a human expert, update the parameters for classification. Settings and/or other configuration information, for example, can be guided by learned use of settings and/or other configuration information, and, as a system is used more (e.g., repeatedly and/or by multiple users), a number of variations and/or other possibilities for settings and/or other configuration information can be reduced for a given situation.

An example deep learning neural network can be trained on a set of expert classified data, for example. This set of data builds the first parameters for the neural network, and this would be the stage of supervised learning. During the stage of supervised learning, the neural network can be tested whether the desired behavior has been achieved.

Once a desired neural network behavior has been achieved (e.g., a machine has been trained to operate according to a specified threshold, etc.), the machine can be deployed for use (e.g., testing the machine with “real” data, etc.). During operation, neural network classifications can be confirmed or denied (e.g., by an expert user, expert system, reference database, etc.) to continue to improve neural network behavior. The example neural network is then in a state of transfer learning, as parameters for classification that determine neural network behavior are updated based on ongoing interactions. In certain examples, the neural network can provide direct feedback to another process. In certain examples, the neural network outputs data that is buffered (e.g., via the cloud, etc.) and validated before it is provided to another process.

Deep learning machines using convolutional neural networks (CNNs) can be used for image analysis. Stages of CNN analysis can be used for facial recognition in natural images, computer-aided diagnosis (CAD), etc.

High quality medical image data can be acquired using one or more imaging modalities, such as x-ray, computed tomography (CT), molecular imaging and computed tomography (MICT), magnetic resonance imaging (MRI), etc. Medical image quality is often not affected by the machines producing the image but the patient. A patient moving during an MRI can create a blurry or distorted image that can prevent accurate diagnosis, for example.

Interpretation of medical images, regardless of quality, is only a recent development. Medical images are largely interpreted by physicians, but these interpretations can be subjective, affected by the condition of the physician's experience in the field and/or fatigue. Image analysis via machine learning can support a healthcare practitioner's workflow.

Deep learning machines can provide computer aided detection support to improve their image analysis with respect to image quality and classification, for example. However, issues facing deep learning machines applied to the medical field often lead to numerous false classifications. Deep learning machines must overcome small training datasets and require repetitive adjustments, for example.

Deep learning machines, with minimal training, can be used to determine the quality of a medical image, for example. Semi-supervised and unsupervised deep learning machines can be used to quantitatively measure qualitative aspects of images. For example, deep learning machines can be utilized after an image has been acquired to determine if the quality of the image is sufficient for diagnosis. Supervised deep learning machines can also be used for computer aided diagnosis. Supervised learning can help reduce susceptibility to false classification, for example.

Deep learning machines can utilize transfer learning when interacting with physicians to counteract the small dataset available in the supervised training. These deep learning machines can improve their computer aided diagnosis over time through training and transfer learning. However, a larger dataset results in a more accurate, more robust deployed deep neural network model that can be applied to transform disparate medical data into actionable results (e.g., system configuration/settings, computer-aided diagnosis results, image enhancement, etc.).

Alternatively or in addition, deep learning and/or machine learning can be implemented via a neural network to process incoming data to generate an output and benefit from feedback to improve its processing. A “recurrent neural network” or “RNN” is a type of neural network in which nodes or cells include loops to allow information to persist over time. Thus, the RNN can leverage reasoning about previous events to inform subsequent processing. In an RNN, a memory or other internal state is used to process input sequence(s) in an element-by-element process wherein an output for each element is dependent on the output of previous and/or other elements (e.g., a directed graph driving a sequence).

“Long short-term memory” networks or “LSTM” networks are RNNs designed to handle long-term dependencies. Generally, LSTM networks are organized into cells and gates which interact to optimize the output of the network. Information from outside the processing of the current element (e.g., information from previous elements) is stored in gated cells. These gates release information based on the weight of the gates, which are adjusted and optimized during the training phase of the AI. In an LSTM network (or its pared-down variant gated recurrent unit network), the nodes or cells in the network have storage and an associated stored state under control of the neural network to aid in establishing correlations and processing input data.

A plurality of deep learning frameworks can be used to evaluate and perform tasks. However, these frameworks have deficiencies and problems that have gone unaddressed. For example, deep learning frameworks such as TensorFlow and PyTorch are highly flexible, but these frameworks require a steep learning curve. In contrast, Ludwig and NiftyNet provide user-friendly environments by abstracting out low-level functionalities, but these frameworks are inflexible. Certain examples provide a FastEstimator deep learning framework, which concurrently supports by design both novice and advanced users with both its flexibility and ease of use.

As the complexity of state-of-the-art deep learning models increases by the month, implementation, interpretation, and traceability become ever-more-burdensome challenges for artificial intelligence (AI) systems. Several AI frameworks have risen in an effort to stem this tide, but the steady advance of the field has begun to test the bounds of their flexibility, expressiveness, and ease of use. To address these concerns, certain examples provide a radically flexible high-level open source deep learning framework for both research and industry.

For example, a high-level open source deep learning framework (e.g., referred to herein as FastEstimator) provides correlation, analysis, and generation of AI models for research, industry, etc. The framework reduces an amount of effort required to create and train complex neural networks without sacrificing flexibility and expressiveness, for example. Moreover, the FastEstimator framework provides convenient utility tools to enhance model interpretability and traceability. Certain examples provide application templates to leverage the framework as ready-made end-to-end solutions for AI modeling, analysis, data processing, etc.

Researchers and experts require ultimate flexibility from AI systems since their goal is to explore new ideas and discover uncharted territory in AI. Frameworks such as TensorFlow, Caffe, MXNet, CNTK, and PyTorch gained the favor of expert users because they make few assumptions regarding user behaviors and allow users to control fine-grained details by building experiments from the ground up. However, building AI from scratch may result in unnecessary verbosity and redundant efforts. Therefore, a framework that preserves flexibility while removing these redundancies is preferable for experts and researchers and improves their productivity.

Entrepreneurs, beginners, and enterprise users tend to favor AI systems that have lower learning curves and faster time to deployment. High-level frameworks such as Keras, Gluon, fastai, and Ludwig are examples of such systems. A benefit of a higher-level framework is ease of use. However, simplicity comes at the expense of flexibility. Furthermore, as more and more new ideas in AI have proven useful in real-world applications, high-level frameworks are not evolving fast enough to serve the industry's interests in these ideas. For example, there are few high-level frameworks that provide flexible support for generative adversarial network (GAN) applications and progressive training schemes. As a result, gaps are forming between state-of-the-art (SOTA) and ease of use.

For at least these reasons, certain examples provide an open source high-level deep learning framework (FastEstimator) to bridge the gap by providing a simple yet flexible interface for implementing AI. For researchers, the framework can continuously monitor the latest advancements in AI to provide an easy and flexible interface. For industry, the framework can enable generation of more AI products and shorten the product development cycle.

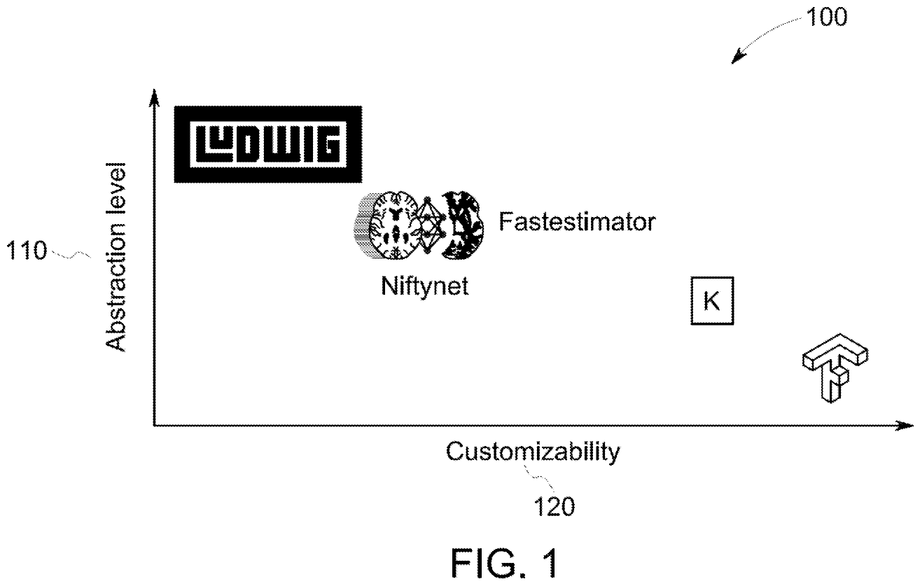

FIG. 1 illustrates an example comparison 100 of artificial intelligence (AI) frameworks based on abstraction level 110 and customizability 120. As shown in the example of FIG. 1, Ludwig has a higher level of abstraction but less customizability, while TensorFlow is highly customizable but offers less abstraction. FastEstimator provides a middle ground with respect to both abstraction level 110 and customizability 120. FastEstimator provides a high-level AI framework built on TensorFlow with an application programming interface (API) and functionalities for a variety of user bases, for example.

For example, Ludwig, developed by Uber for AI amateurs, is code free and difficult to customize. Keras is a high-level framework, implemented as Keras or TF.Keras, that is easy to customize. NiftyNet only supports medical imaging-related tasks (e.g., image segmentation and classification) and is difficult to customize. In contrast, the FastEstimator AI framework is semi-code free, built on top of TensorFlow and TF.Keras, easily usable for most users. For example, FastEstimator provides a Keras model architecture with a TensorFlow train engine. FastEstimator allows easy customization and provides a general purpose task framework with a healthcare specialization.

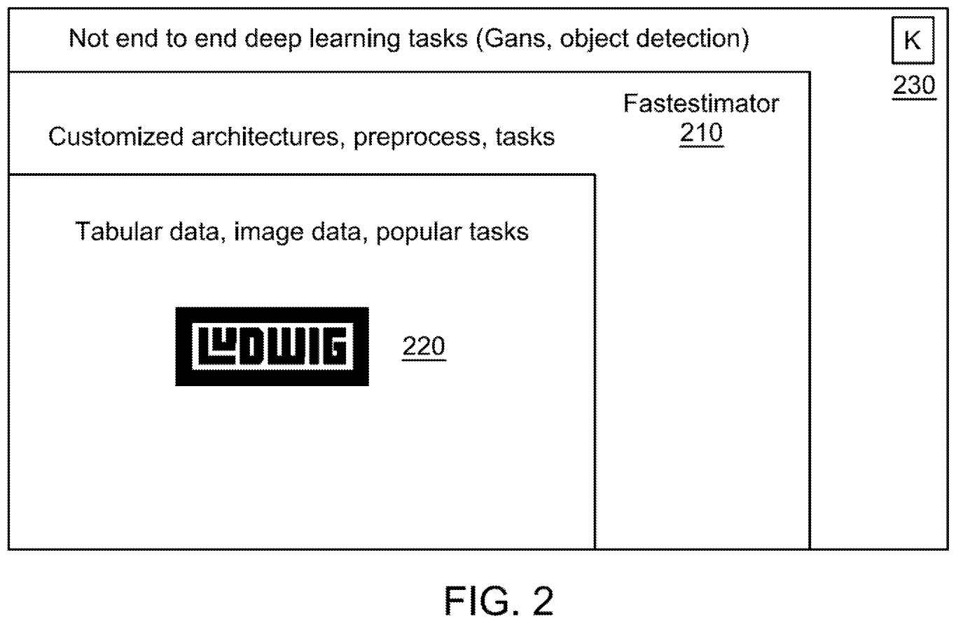

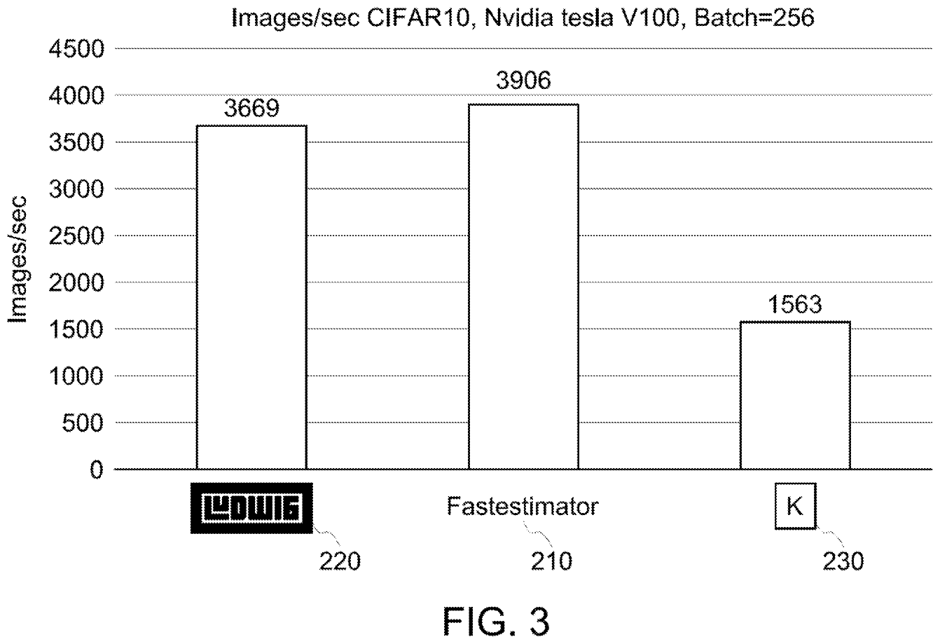

FIG. 2 illustrates an example comparison in supported functionality between FastEstimator 210 and other AI frameworks 220, 230. As shown in the example of FIG. 2, an area of the associated rectangle corresponds to a number of tasks that the AI framework 210-230 can handle. For example, the Ludwig framework 220 supports processing of tabular data, image data, and popular tasks. The Keras framework 230 provides support except for end-to-end deep learning tasks such as GANs, object detection, etc. The FastEstimator framework 210 sits in the middle and supports customized architectures, pre-processing, tasks such as synthetic image generation, object detection, etc. FIG. 3 provides an example training speed comparison among frameworks 210, 220, 230.

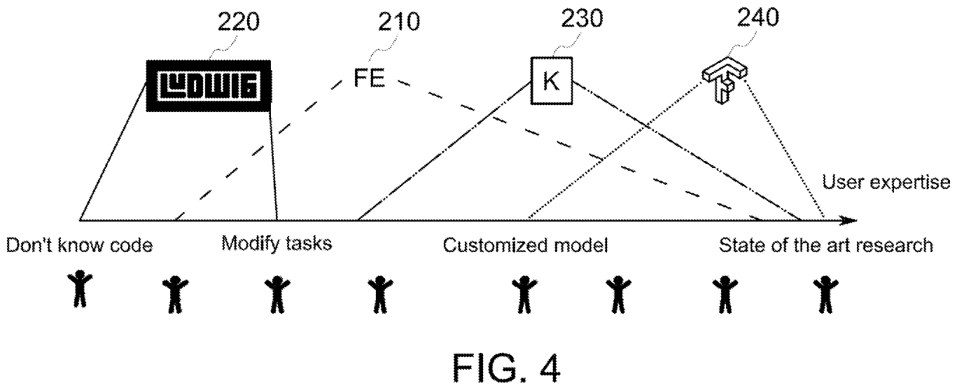

As illustrated in the example of FIG. 4, triangles formed from the lines indicate a user population covered by the respective AI framework 210-240. For an amateur user who wants to adapt existing tasks to his/her own, Ludwig 220 and FastEstimator 210 are easy, while Keras 230 and TensorFlow 240 are difficult to use, for example. For an intermediate user who wants custom pre-processing, layers, etc., Ludwig 220 is difficult, and FastEstimator 210 is easy, while Keras 230 and TensorFlow 240 are easy in some respects and difficult to use in other respects, for example. For an advanced user who wants to conduct state of the art research, Ludwig 220 is hard and FastEstimator 210 is easy in some respects and difficult to use in other respects, while Keras 230 and TensorFlow 240 are easy to use. As shown in the example of FIG. 4, Ludwig 220 provides a platform for users who do not know how to code and are looking for basic modification of tasks. FastEstimator 210 provides a framework for users who want to modify tasks, develop customized models, and do some state of the art research. Keras 230 provides a framework for users who want customized models and state of the art research, and TensorFlow 240 provides a framework for customized models and state of the art research.

Thus, certain examples provide a FastEstimator AI framework that is easy to use and customizable for most AI tasks. FastEstimator can include a healthcare specialization including a set of templates, architectures, and/or weights for healthcare-related tasks such as object detection, synthetic image generation, etc. FastEstimator enables fast training speed including an efficient pipeline, fast convergence method, multi-general processing unit (GPU) training, mixed precision training, etc., as well as security and encryption support, for example. FastEstimator is semi-code free and can be customized using Python and/or other programming language/script, for example. FastEstimator provides pre-trained models, tabular data, image classification, image segmentation, synthetic image generation via a generative adversarial network (GAN), etc. While other AI tools are inflexible and limited in scope, with users limited to only a specific set of models provided, FastEstimator balances ease of use for novice users and customization for advanced practitioners using a semi-code free interface, for example.

In certain examples, the FastEstimator framework is a high-level deep learning framework built on TensorFlow and/or other symbolic math library for machine learning applications. The library allows the framework to leverage application programming interfaces (APIs) for AI model design and training such as tf.keras, etc. For training, the FastEstimator framework extends beyond the functionality of the underlying API (e.g., tf.keras, etc.) by enabling more complex training schemes such as multi-model training, multi-task training, and progressive training.

Using the FastEstimator framework, three main APIs can be leveraged for a plurality of deep learning tasks. Additionally, an operator generator enables definition of a complex computational graph in a concise manner using one or more computational operators. A trace generator provides further control over the training loop by inserting event functions in a training loop, propagating event information between traces in training, computing metrics regarding training, etc. Using these generators (e.g., application program modules, etc.), the framework can significantly reduce the effort required to implement several deep learning tasks.

The framework is also easy to scale. In other solutions, distributed training in many deep learning frameworks requires non-trivial effort on the user's side. For example, users are expected to understand device communication patterns and rewrite their workflows to be distribution-aware. Using the FastEstimator framework, however, modules are designed to be distribution-friendly such that users can scale their training and evaluation across multiple devices without change of code.

In addition to the training APIs, the FastEstimator framework offers useful AI utility functions to facilitate prototyping and production processes. For example, a model interpretation module includes visualization tools such as feature uniform manifold approximation and projection (UMAP), saliency maps, and caricature maps to help users build more robust models. Utility modules have full compatibility with TensorBoard and/or other machine learning visualization and tooling software. The framework can also provide tools for automatic report generation, other documentation, etc.

Some frameworks provide a model “zoo” or collection, which allows users to import pre-built model architectures and weights. However, it is often the case that the true complexity of implementing a new idea lies more on the data pipeline and training loop than on the model architecture itself. For at least this reason, the FastEstimator framework provides an application hub, which functions as a place to showcase different end-to-end AI applications. Each template in the application hub has step-by-step instructions to help ensure users can easily build new AI applications with their own data, for example.

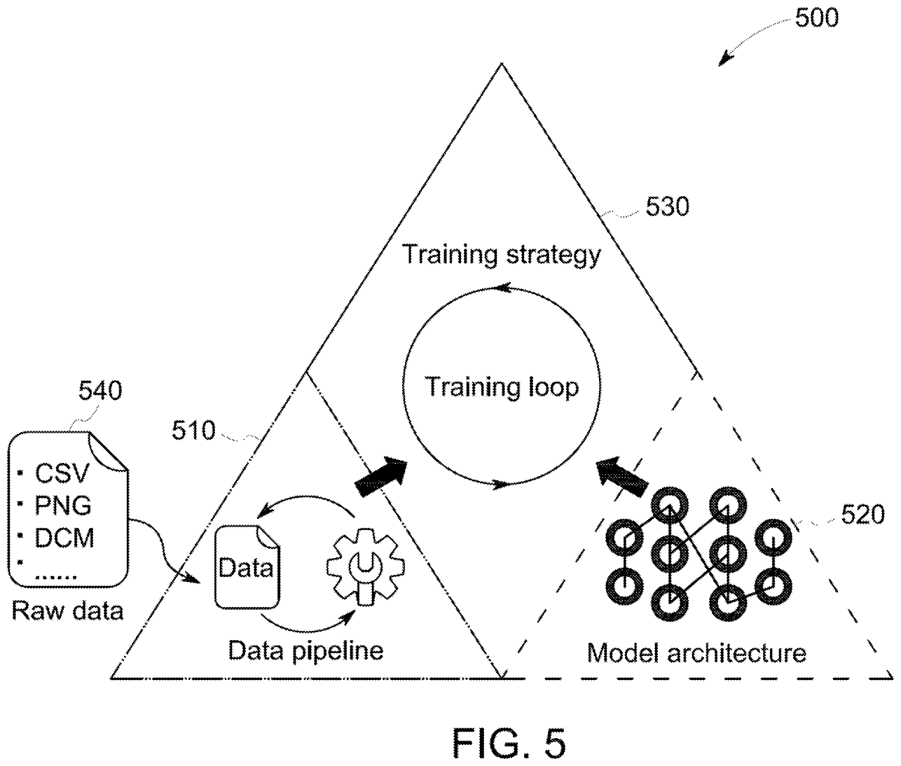

FIG. 5 illustrates example components and associated workflow of a FastEstimator AI framework 500. The example framework 500 is a semi-code free deep learning framework with ease of use and fast training that can cater to the needs of a wide range of users in healthcare and other areas.

The FastEstimator framework 500 includes a data pipeline 510, a model architecture 520, and a training loop 530. Each component 510-530 of the example framework 500 is designed with object-oriented principles such that operations are easily customizable for specific tasks. The data pipeline 510 enables efficient training, for example. The FastEstimator framework 500 automatically fine-tunes the data pipeline 510 to maximize and/or otherwise improve resource utilization. In addition, the FastEstimator framework 500 supports model architecture 520 definition such as using TensorFlow-Keras, etc., to preserve simplicity and flexibility. Both model 520 and pipeline 510 are sent to the training loop 530 for users to customize training and create deep learning models. Raw data 540 can be introduced to the data pipeline 510 and processed according to the model architecture 520, used by the training loop 530, etc. When comparing the training speed of FastEstimator against Ludwig and NiftyNet on classification and segmentation tasks, for example, FastEstimator is 2.95 times faster than NiftyNet on segmentation tasks and 1.06 times faster than Ludwig on classification tasks, for example.

The FastEstimator framework 500 provides a modular API layout design. The FastEstimator 500 divides deep learning tasks into the data pipeline 510, model architecture 520, and training loop 530. Each component 510-530 is designed with object-oriented principles such that operations are easily customizable for specific tasks. An advantage of the modular design of the FastEstimator framework 500 is that a user can easily customize deep learning tasks without making source code changes. The modular layout design in the form of the data pipeline 510, model 520, and training strategy 530 is unique and does not exist in any other AI frameworks.

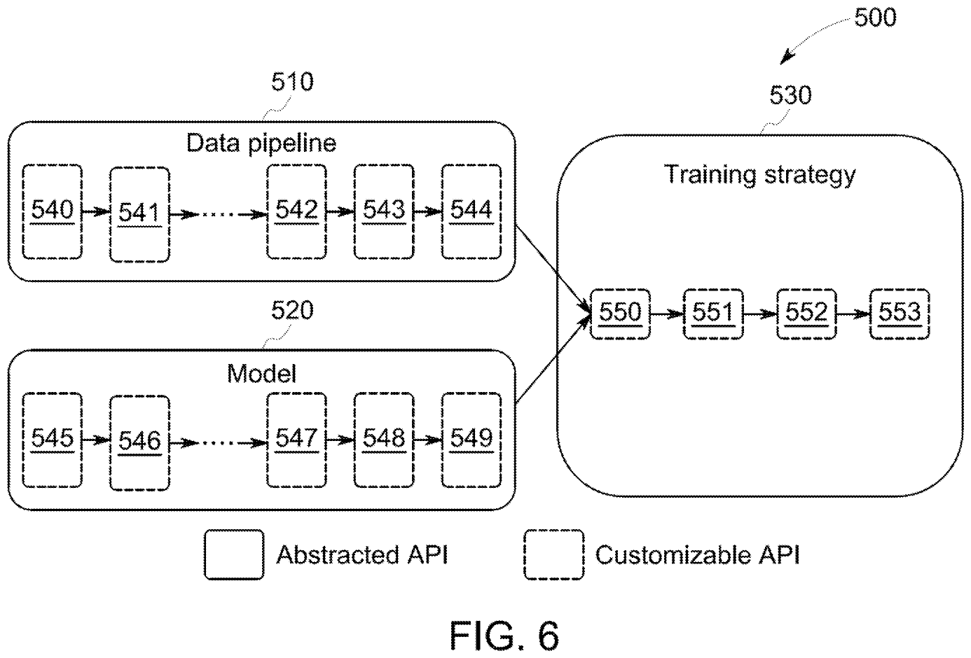

FIG. 6 illustrates an example implementation of the modular design of the FastEstimator AI framework 500 including the data pipeline 510, model architecture 520, and training loop 530. Each of the data pipeline 510, model architecture 520, and training loop 530 includes an abstracted API as well as a plurality of customizable APIs, for example. As shown in the example of FIG. 6, each abstracted API 510-530 masks a plurality of customizable APIs 540-553 which leverage underlying framework to provide functionality available externally to generate and provide AI model(s) 520 and leverage input data through the data pipeline 510 to drive a training strategy 530 for the model(s) 520 using the data pipeline 510.

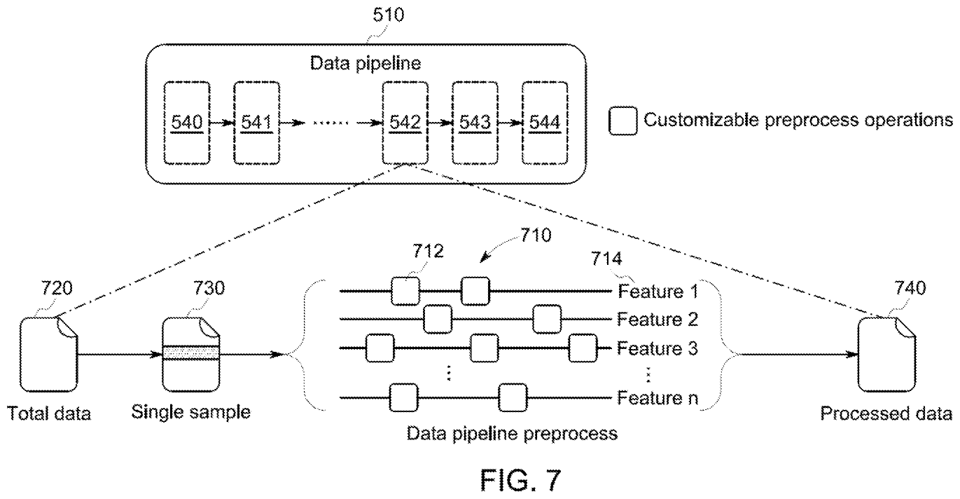

FIG. 7 illustrates a further example of the data pipeline 510 and its modular data pipeline preprocessing implementation. A preprocessor 710 of the example data pipeline 510 is designed to handle complex feature preprocessing operations. Each preprocessing operation is considered as a block 712 that acts on single or multiple feature(s) 714. Each block 712 is easily customizable to support user's own preprocessing tasks. The example implementation of FIG. 7 allows users to bring their own customized preprocessing tasks without making any source code change to the framework 500. Such a modular data pipeline preprocessor 710 does not exist in any other AI framework. Thus, from a total data set 720, a single sample 730 can be provided to the preprocessor 710 to be preprocessed according to one or more operations 712 acting on feature(s) 714 of the data sample 730 to generate processed data 740.

Thus, while most application-based AI frameworks such as Ludwig and NiftyNet suffer from the drawbacks of being inflexible and hard to customize, certain examples provide a flexible, customizable FastEstimator framework. The root cause of the inflexibility in prior systems comes from failure in API abstraction design to support specific use cases. Most APIs of such AI frameworks cannot be easily customized without changing the underlying source code. FastEstimator, however, allows easy customizable without extensive source code editing. Additionally, FastEstimator's modular API layout design can effectively abstract out essential components of any end-to-end deep learning task and simplify the deep learning experience for novice users. Further, FastEstimator's modular design exposes all of its components to users through its highly customizable API. As a result, this flexibility enables intermediate and advanced users to fully customize their deep learning tasks.

Compartmentalization of the data pipeline, model architecture, and trainer allows elements of each component to be individually customized such as with custom user models, preprocessing functions, training algorithms, etc. Customization of models, processing functions, training loops, etc., can be targeted for specific application(s), domain(s), etc., and still operate within the general FastEstimator framework or platform, for example. Compartmentalization allows a variety of models to be accommodated by the FastEstimator framework as well as customization of preprocessing operations on input data. For example, the framework can train on synthetic image data, image classification, etc., and can allow a user to customize his or her own pre-processing operation for each specific feature associated with a processing/pre-processing task. The data pipeline can then grab feature(s) for further processing.

Thus, certain examples provide an AI framework for model training, computing tasks, etc. The example framework can be implemented on a cloud-based server, a backend computer, an edge device or gateway, a local computer, etc. The example framework can perform pre-processing operations to prepare for execution of one or more pre-processing and/or processing tasks.

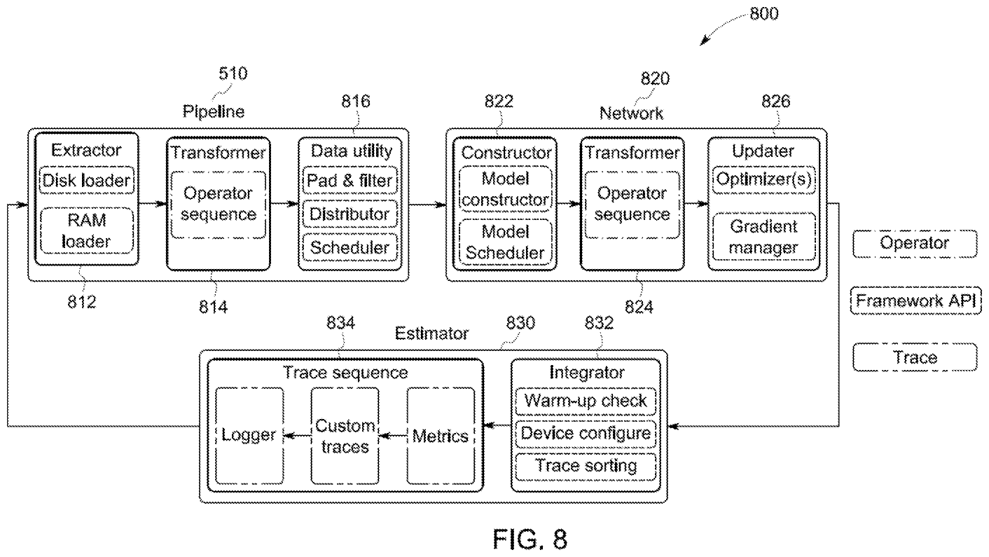

As shown in FIG. 8, certain example deep learning training workflows and associated apparatus 800 involve three primary components: a data pipeline 510, a network 820, and an optimization strategy implemented by an estimator 830. The data pipeline 510 extracts data from disk to memory such as RAM, etc., performs transformations, and then loads the data onto a device, for example. The network 820 stores trainable and differentiable graphs, for example. The optimization strategy of the estimator 830 combines the data pipeline 510 and the network 820 in an iterative process, for example. Each of these components represents an API in the example FastEstimator framework 800 shown in the example of FIG. 8. These elements and associated API enable the example framework 800 to be used for a variety of deep learning tasks.

The example data pipeline 510 can include/implement an Extraction-Transformation-Load (ETL) process using an extractor 812, a transformer 814, and a data utility 816. The extractor 812 can take data from disk, RAM, other memory, etc., with features being either paired or unpaired (e.g., domain adaptation, etc.,). The transformer 814 builds graphs for preprocessing. The data utility 816 provides support for scenarios such as imbalanced training, feature padding, distributed training, progressive training, etc.

The example network 820 manages trainable AI models. As shown in the example of FIG. 8, a constructor 822 builds model graphs and creates timestamps on these graphs in the case of progressive training, for example. A transformer 824 then connects different pieces of model graphs and non-trainable graphs together. An updater 826 tracks and applies gradients to each trainable model.

For example, a gradient captures partial derivatives of a multi-variable function associated with the graphs (e.g., in a vector-valued function, etc.). A derivative represents a rate of change or slope of the function at a given point. Using partial derivatives, the gradient indicates a direction in which an associated function increases the most (e.g., a direction of steepest ascent of a variable included in the function). If there are multiple variables in the function, there will be a corresponding number of partial derivatives in the gradient vector (e.g., an n-variable function provides an n-dimensional gradient vector). A gradient descent moves in a direction opposite to the gradient to reduce or minimize deviation, loss, or other error in the particular variable. The gradient can be used to tune weights on inputs, connections, nodes, etc., in a network model, for example. Gradients can be determined, monitored, and adjusted to converge to or otherwise stabilize model weights and/or other model configuration parameters, for example.

In the example apparatus 800, the estimator 830 is responsible for providing and maintaining an AI model training loop. Before training starts, a “smoke test” is performed on all graphs to detect potential run-time errors as well as to warm up the graph for faster execution. The estimator 830 then proceeds with training, generating any user-specified output along the way. The example estimator 830 includes an integrator 832 that checks model graphs, sorts connectivity/execution traces, and configures device(s) to which models are to be deployed, for example. The example estimator 830 includes a trace sequence 834 including metrics for model execution, execution traces represented in model graphs, and a logger to generate and/or otherwise capture a record or log of AI model construction, execution, etc., which can be provided as feedback into the data pipeline 510 to be loaded by the extractor 812, transformed by the transformer 814, etc.

In certain examples, the pipeline 510 and network 820 include a sequence of operators to facilitate organization of data, building and operation of AI models, etc. The estimator 830 includes a sequence of traces representing model execution, for example. Operators and traces are described further below.

In certain examples, deep learning APIs enable complex graph building with less code. As such, layers (e.g., blocks and code modules) can be used to simplify network definition. However, as model complexity increases, layer representations may become undesirably verbose (e.g., when expressing multiple time-dependent model connections, etc.). Therefore, certain examples provide a higher level abstraction for layers, referred to as an operator, to achieve code reduction without losing flexibility.

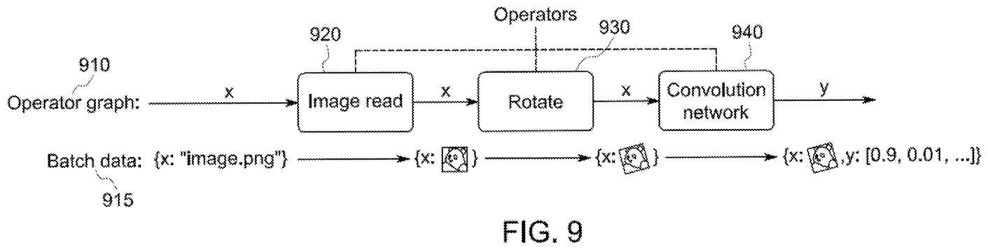

An operator represents a task-level computation module for data (e.g., in the form of key:value pairs), that can be trainable or non-trainable. In certain examples, each operator has three components: input key(s), transformation function, and output key(s). The execution flow of a single operator involves: 1) take the value of the input key(s) from batch data, 2) apply transformation functions to the input value, and 3) write an output value back to the batch data with output key(s). FIG. 9 shows how an example deep learning application can be expressed as a sequence of operators.

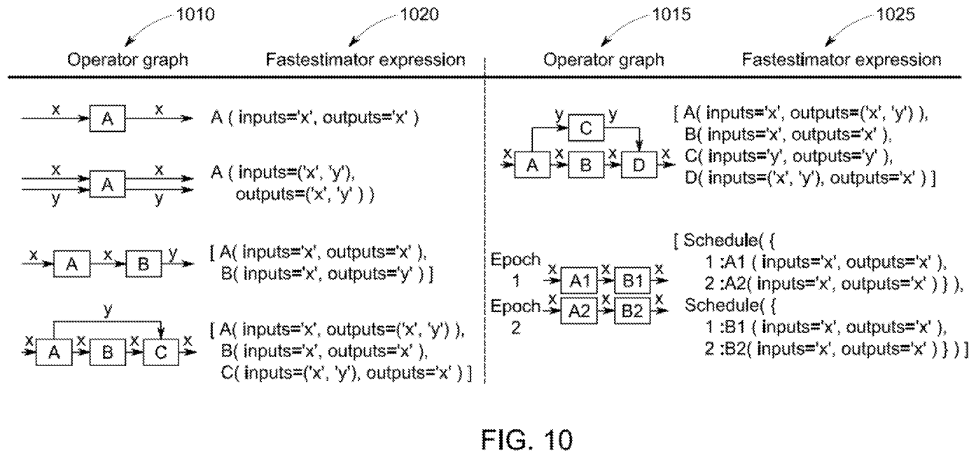

As shown in the example of FIG. 9, an operator graph 910, corresponding to batch data 915, is formed using an image x provided to an image read operator 920 to read the image data and determine image content. The read image is then provided to a rotate operator 930 to rotate the image a determined or predetermined amount (e.g., certain degrees, etc.). The rotated image data is then provided to a convolution network operator 940 to process the rotated image data to determine information about the image data such as intensities, coordinates, other expression, etc. Thus, using the operators 920-940, the example FastEstimator platform provides concise operator expressions to facilitate efficient construction of various graph topologies. Using operators 920-940, complex computational graphs can be built using a few lines of code. For example, FIG. 10 shows example operator graphs 1010, 1015 and associated expressions 1020, 1025.

Some APIs offer two separate modules: metrics and callbacks (e.g., hooks and handlers). However, in certain examples, the FastEstimator framework unifies metrics and callbacks into traces. As a result, several limitations introduced by separating metrics and callbacks can be overcome. Metrics are quantitative measures of model performance and are computed during a training or validation loop. From the implementation perspective, APIs tend to implement metrics as a built-in computational graph with two parts: a value and an update rule. While this pre-compiled graph enables faster computation, it also limits the choice of metrics. For example, some domain-specific metrics are not easily expressed as a graph or require running external post-processing libraries. Furthermore, the benefit offered by pre-compiling metric graphs is not significant because these calculations only account for a small portion of the system's total computation.

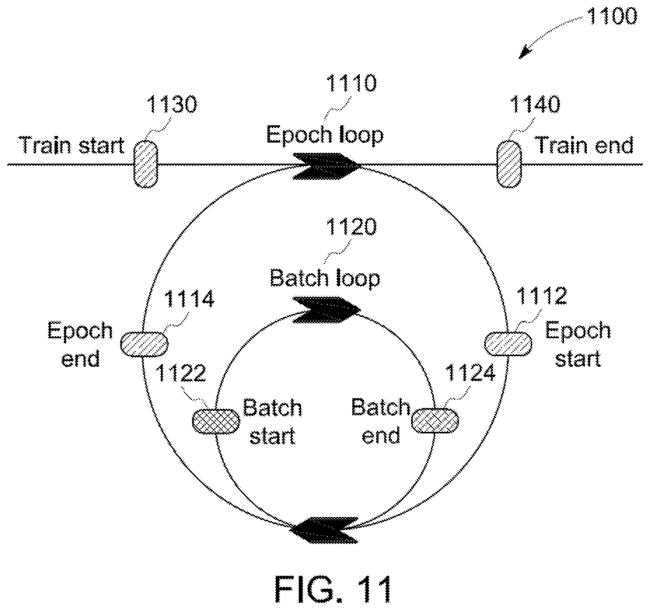

Callbacks are software modules that contain event functions like on_epoch_begin and on_batch_begin, which allow users to insert custom functions to be executed at different locations within the training loop, such as shown in the example of FIG. 11. In terms of implementation, since metrics and callbacks are separate, callbacks in most frameworks are not designed to have easy access to batch data. As a result, researchers may have to use less efficient workarounds to access intermediate results produced within the training loop. Moreover, callbacks are not designed to communicate with each other, which adds further inconvenience if a later callback needs the outputs from a previous callback.

The example of FIG. 11 shows events within a training loop 1100. The example training 1100 includes an epoch loop 1110 and a batch 1120 between a training start 1130 and a training end 1140. Each loop 1110, 1120 has start 1112, 1122 and an end 1114, 1124. When both loops 1110, 1120 have been completed, the training ends, for example.

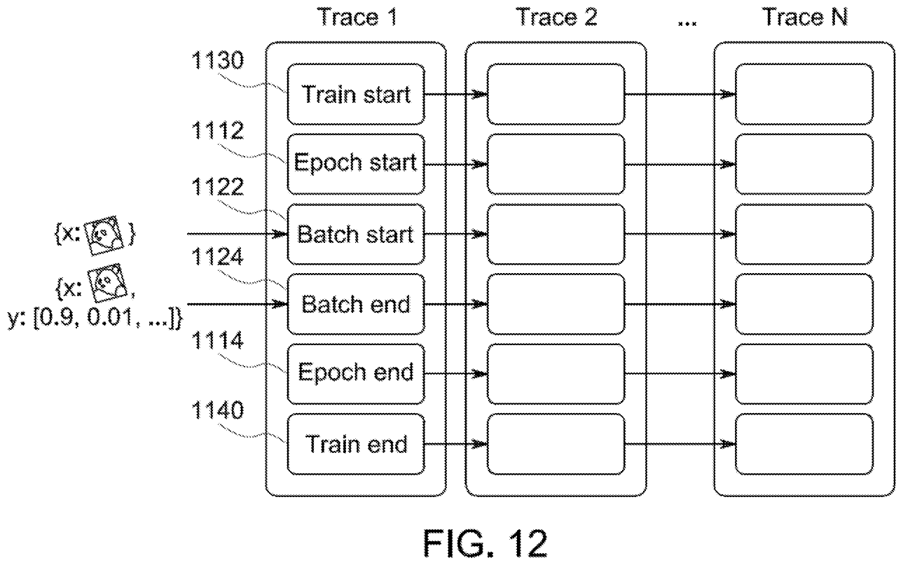

In the example framework, a trace is a unification of metrics and callbacks. The trace preserves event functions in callbacks and overcomes limitations in callbacks. For example, traces have easy access to batch data directly from the batch loop. Additionally, each trace can pass data to later traces to increase re-usability of results as shown in the example of FIG. 12. Further, metric computation can leverage batch data directly without a graph. Metrics can be accumulated through trace member variables without update rules, for example. These improvements provided by traces have enabled many new functionalities that are not easily achieved with conventional callbacks. For example, a model interpretation module can be enabled by easy batch data access. Furthermore, a trace has access to API components such that changing model architecture or data pipeline within the training loop. Such API access can be used to unlock support for Meta-Learning, Reinforcement Learning (RL), and AutoML algorithms, for example.

For example, as shown in FIG. 12, batch data from operator(s) 920-940 can be provided to a first trace, which begins training and processes an epoch loop 1110 and a batch loop 1120 within the epoch loop 1110. Metrics gathered from the first trace can be passed to a second trace, and epoch and batch training metrics can be propagated through a series of traces 1 to N, for example. As such, events occurring within a training loop for trace can be communicated to other traces, for example.

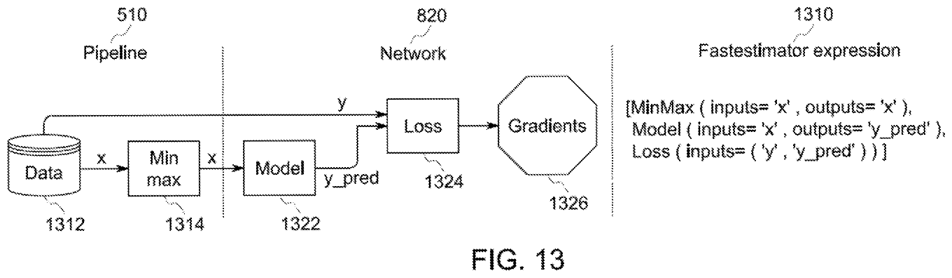

FIGS. 13-17 depict example operator expressions for various deep learning tasks to be implemented using the FastEstimator platform 500, 800. FIG. 13 shows an example implementation of image classification using the data pipeline 510, network 820, and estimator 830. As shown in the example of FIG. 13, the data pipeline 510 and network 820 can be configured to generate an expression 1310 of an AI model function (e.g., image classification, etc.). For example, batch data 1312 provides image and/or other data including a key x and a label key y. The key x is input to a min/max function 1314 to normalize the input image and/or other data including the key x. The normalized data is provided to an AI model 1322 in the network 820, which executes a forward pass of the normalized data to generate a predictive output for y_pred). This predicted value, y_pred, is compared to an actual value of y taken from the batch data 1312 using a loss function 1324 to calculate gradients 1326 between y and y_pred and perform back propagation, for example.

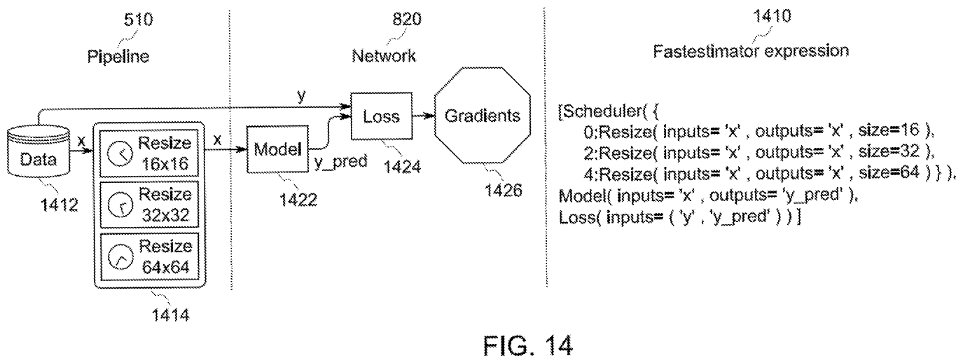

FIG. 14 shows an example of image classification with progressive resizing using the example FastEstimator platform 500, 800. The data pipeline 510 and the network 820 of the example of FIG. 14 can be configured to generate an expression 1410 of an AI model function. For example, batch data 1412 provides image and/or other data including a key x and a label key y. The key x is input to a progressive resizer 1414 to resize and increase image resolution of the input image and/or other data including the key x (e.g., 16×16, 32×32, 64×64, etc.). The resized data is provided to an AI model 1422 in the network 820, which executes a forward pass of the normalized data to generate a predictive output for y (y_pred). This predicted value, y_pred, is compared to an actual value of y taken from the batch data 1412 using a loss function 1424 to calculate gradients 1426 between y and y_pred and perform back propagation, for example. As shown in the example of FIG. 14, the data 1412 begins with a low resolution image and the resizer 1414 gradually increases the image resolution as training progresses. In the example of FIG. 14, image resolution is increased by the progressive resizer 1414 by a factor of two on epochs 2 and 4.

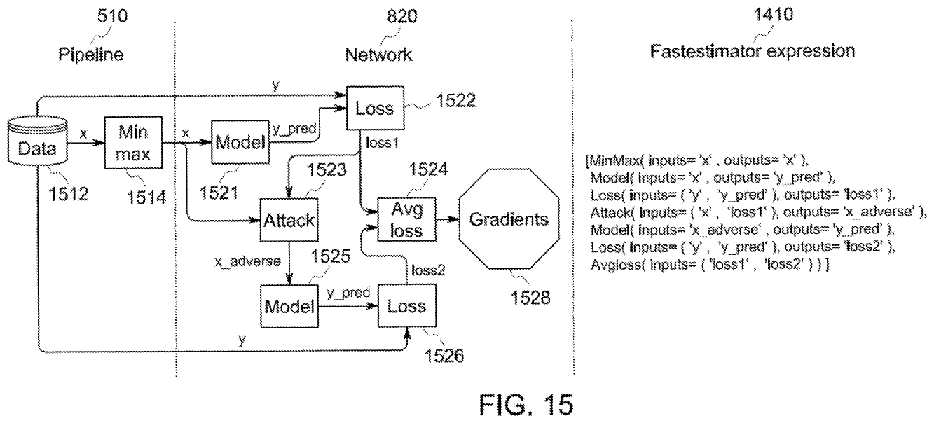

FIG. 15 shows an example of image classification with adversarial training using the example FastEstimator platform 500, 800. The data pipeline 510 and the network 820 of the example of FIG. 15 can be configured to generate an expression 1510 of an AI model function. For example, batch data 1512 provides image and/or other data including a key x and a label key y. The key x is input to a min/max function 1514 to normalize the input image and/or other data including the key x. The normalized data is provided to an AI model 1521 in the network 820, which executes a forward pass of the normalized data to generate a predictive output for y (y_pred). This predicted value, y_pred, is compared to an actual value of y taken from the batch data 1512 using a loss function 1522. An output of the loss function 1522 is a first loss, which is used to form an adversarial attack 1523 to perturb the input image x to form an image x_adverse. The first loss output is also provided to an average loss calculator 1524. The adversarial attack image, x_adverse, is provided to a second AI model 1525 to calculate another predicted value, y_pred, to be compared, using a second loss function 1526, to the actual value of y from the batch data 1512. A second loss output is also fed into the average loss calculator 1524 to be used with the first loss output to determine gradients 1528 between y and y_pred and perform back propagation, for example. As such, adding four operators 1523-1526 adds adversarial training to the model 1521 and makes the model 1521, 1525 more robust to future adversarial attacks, for example. Adversarial training can be added to any model using operators in the example framework 500, 800, for example.

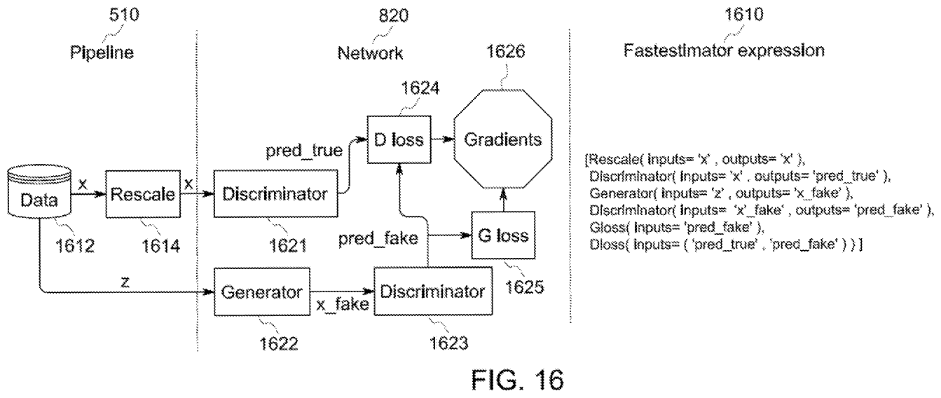

FIG. 16 shows an example of image generation with a deep convolution (DC) generative adversarial network (GAN) implemented using the example FastEstimator platform 500, 800. For multi-model training such as DC-GAN, different losses can be associated with different models. Gradients are calculated with respect to each loss, and the system performs updates for each model, for example. As shown in the example of FIG. 16, the data pipeline 510 and the network 820 are configured to generate an expression 1610 of an AI model function. For example, batch data 1612 provides a real image x and random noise z. The real image x is rescaled using a rescaler 1614 and input to a discriminator 1621. The random noise z is provided to a generator 1622. The discriminator 1621 processes the rescaled image x and predicts that the rescaled image x is true (pred_true). The generator 1622 processes the random noise z to generate a fake image and/or other data, x_fake. The fake image data is provided to a second discriminator 1623, which processes the image data and identifies it as fake, pred_fake. Output of the discriminator 1623 is provided to two loss functions—a discriminator loss function 1624, D Loss, and a generator loss function 1625, G Loss. Gradients 1626 are calculated with respect to each loss 1624, 1625, for example.

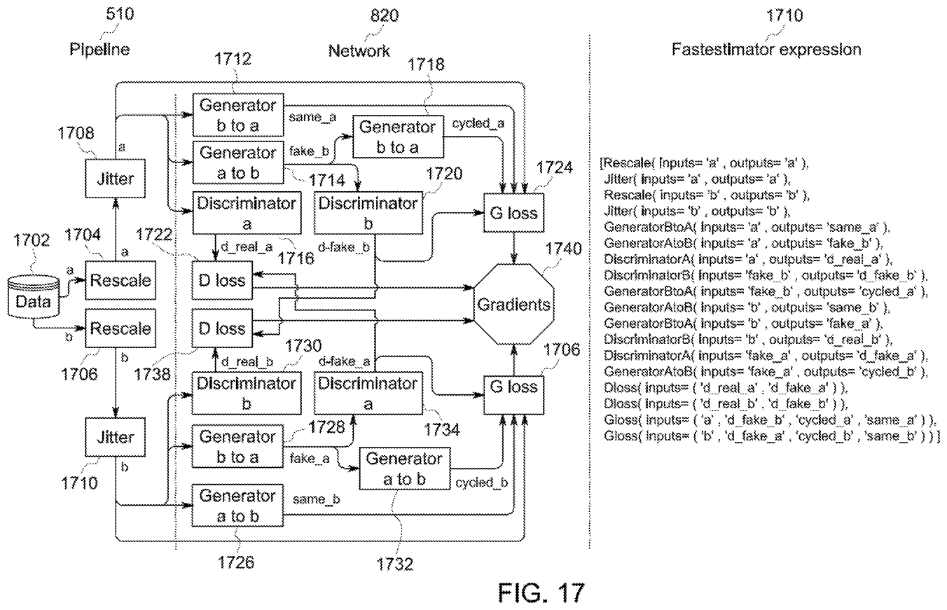

FIG. 17 depicts an example unsupervised, unpaired image translation or generation using a cycle-GAN. In such an example, a user typically needs to define different loss functions that involve outputs of multiple models. Using operators, the FastEstimator framework 500, 800 breaks down and instantiates interactions between different generators and discriminators. As shown in the example of FIG. 17, data 1702 provides unpaired images a and b from two different domains. Image a is provided to a rescale model 1704, and image b is provided to a rescale module 1706 so that images a and b can be processed in parallel. Jitter can be removed from the resized images using a jitter module 1708, 1710.

The jitter-reduced image a is provided to a b-to-a generator 1712, an a-to-b generator 1714, and a discriminator a 1716, for example. The b-to-a generator 1712 provides an output of a (same_a) from the jitter-reduced image input a. The a-to-b generator 1714 produces a fake output b (fake_b) from the jitter-reduced image input a. The fake_b output is provided to another b-to-a generator 1716 to produce a cycled a output. The fake b_output is also provided to a discriminator b 1720, which discriminates the fake_b input to generate an output (d_fake_b). A discriminated output of a (d_real_a) by the discriminator 1716 is provided to a discriminator loss function construct (D Loss) 1722. The jitter-reduced image a, cycled_a, and d_fake_b outputs are provided to a generator loss function construct (G Loss) 1724. A result of D Loss 1722 and G Loss 1724 can be used to generate a gradient 1740.

In parallel, the jitter-reduced image b is provided to an a-to-b generator 1726, a b-to-a generator 1728, and a discriminator b 1730, for example. The a-to-b generator 1726 provides output of b (same_b) from the jitter-reduced image input b. The b-to-a generator 1728 produces a fake output a (fake_a) from the jitter-reduced image input b. The fake_a output is provided to another a-to-b generator 1732 to produce a cycled_b output. The fake_a output is also provided to a discriminator a 1734, which discriminates the fake_a input to generate an output (d_fake_a). A discriminated output of b (d_real_b) by the discriminator 1730 is provided to a discriminator loss function construct (D Loss) 1738. The jitter-reduced image b, cycled_b, and d_fake_a outputs are provided to a generator loss function construct (G Loss) 1736. A result of D Loss 1738 and G Loss 1736 can be used to generate the gradient 1740.

Thus, certain examples provide an AI model-driven framework to develop, train, test, and deploy AI network models (e.g., deep neural networks, deep reinforcement learning networks, GANs, etc., using a plurality of dynamically adaptable operators driving a data pipeline and network to generate estimator expressions. Certain examples allow a plurality of AI models to be connected and developed together for system deployment and usage according to rules, parameters, etc., established using the framework in combination with user, application, and/or other system input.

Additionally, while examples above describe operators, traces, etc., in conjunction with the example AI framework 500, 800, operators and/or traces can be used apart from the example framework 500, 800, for example.

While example implementations are illustrated in conjunction with FIGS. 1-17, elements, processes and/or devices illustrated in conjunction with FIGS. 1-17 can be combined, divided, re-arranged, omitted, eliminated and/or implemented in any other way. Further, components disclosed and described herein can be implemented by hardware, machine readable instructions, software, firmware and/or any combination of hardware, machine readable instructions, software and/or firmware. Thus, for example, components disclosed and described herein can be implemented by analog and/or digital circuit(s), logic circuit(s), programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the components is/are hereby expressly defined to include a tangible computer readable storage device or storage disk such as a memory, a digital versatile disk (DVD), a compact disk (CD), a Blu-ray disk, etc. storing the software (including computer- and/or other machine-readable instructions) and/or firmware.

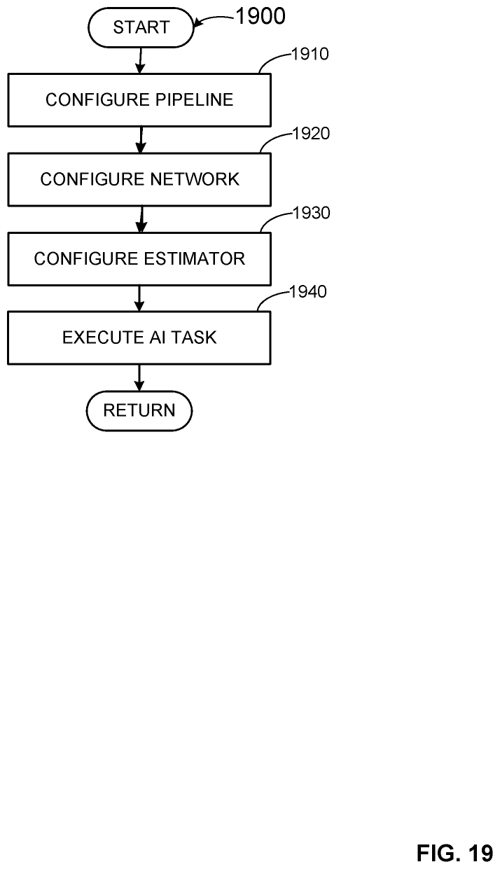

In the examples, the machine readable instructions include a program for execution by a processor such as a processor 1912 shown in the example processor platform 1900 discussed below in connection with FIG. 19. The program may be embodied in machine readable instructions stored on a tangible computer readable storage medium such as a CD-ROM, a floppy disk, a hard drive, a digital versatile disk (DVD), a Blu-ray disk, or a memory associated with the processor 1912, but the entire program and/or parts thereof could alternatively be executed by a device other than the processor 1912 and/or embodied in firmware or dedicated hardware.

As mentioned above, the example process(es) disclosed and described herein may be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine readable storage medium” are used interchangeably. Additionally or alternatively, the example process(es) can be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended. In addition, the term “including” is open-ended in the same manner as the term “comprising” is open-ended.

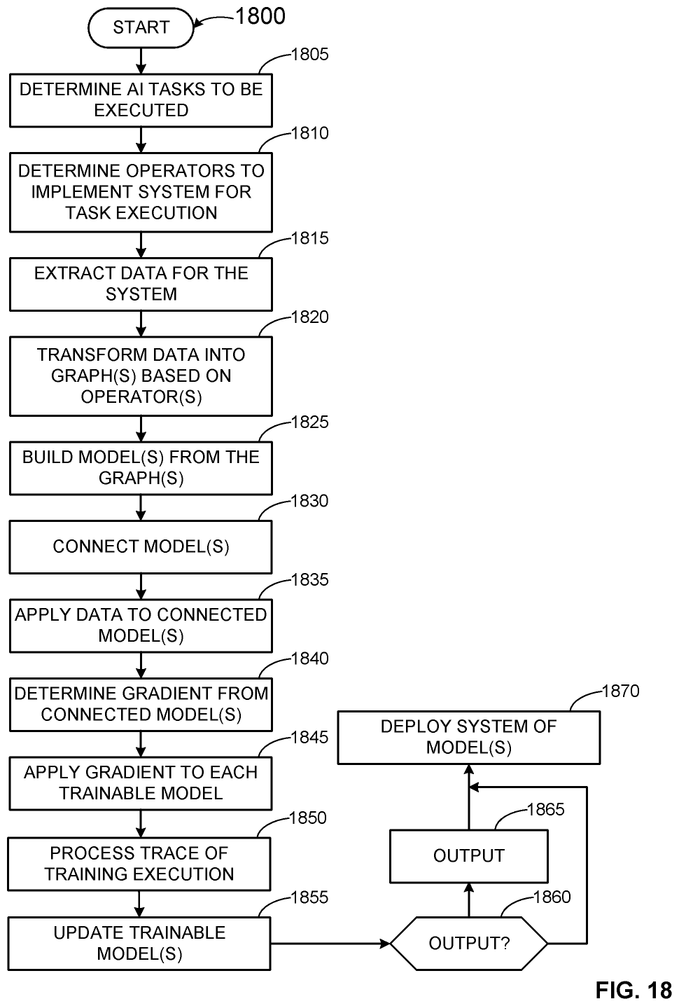

FIG. 18 illustrates a flow diagram of an example method 1800 to dynamically generate AI systems and associated tasks (e.g., deep learning tasks and/or training workflows). At block 1805, one or more machine/deep learning tasks to be executed are determined. For example, task(s) can be determined based on user input and/or other request, an application program instruction, a schedule, a trigger from another system or application, other stored setting, etc. Tasks can include image processing (e.g., image generation, image translation, image classification, etc.), waveform analysis, synthetic data generation, etc., for example.

At block 1810, operators to implement a system for task execution are determined. For example, one or more operators such as a graph operator, image read operator, convolution network operator, other task-level computational/transformational operator, etc. A sequence of operators is used to form a deep learning application, for example.

At block 1815, data is extracted to be provided to the system. For example, batched data (e.g., captured real data and/or generated synthetic data) is leveraged to be applied to the system of operators. The data can be applied for training, testing, adjustment, and/or predictive output, for example.

At block 1820, the data is transformed into one or more graphs based on the operator(s). For example, one or more operators can be used with extracted data to form a computational graph. At block 1825, one or more models are built from the graph(s). For example, one or more deep learning models is formed from the combination of operators defining one or more computational graphs. At block 1830, model(s) are connected. For example, one or more models can be combined to form a network to execute one or more machine learning/deep learning tasks, such as image processing, signal data analysis, synthetic data generation, etc. The network can include trainable models and non-trainable models, for example. A trainable model can be modified based on gradient and/or other feedback from application of real and/or synthetic data to the trainable model. A non-trainable model is generated and fixed, rather than adjustable based on feedback, for example.

At block 1835, data is applied to the connected model(s). For example, data from a real and/or synthetic batched data source is applied to the connected network of models to train and/or test their operation. Events and other trace information can be gathered during the application of data to train the connected network of models. At block 1840, a gradient is determined from the connected model(s). For example, one or more loss functions, trace information, and/or other deviation can be used to form a gradient for the network. The gradient is indicative of an error or difference in outcomes (e.g., expected versus actual, output from model A versus output from model B, etc.) that can be used to adjust model parameters/settings/weights, etc.

At block 1845, the gradient is applied to each trainable model in the system. For example, the deviation can be used to adjust network model weights, connections, and/or other settings/parameters. At block 1850, trace information of training execution is processed to analyze execution of the connected model graph in one or more training loops. For example, one or more training loops can be executed using the connected network of model(s), and trace data can be captured for each epoch loop within the training loop and each batch loop within each epoch loop. Trace information can be propagated to a subsequent trace to track events occurring over multiple traces corresponding to multiple training loops to provide feedback for adjustment of trainable model(s), for example. At block 1855, the trainable model(s) are updated based on the gradient, trace, and/or other data. For example, changes made to the trainable model(s) based on the applied gradient information, event(s) extracted from trace(s), and/or other feedback can be instantiated/deployed for use in the network.

At block 1860, if appropriate, an output 1865 can be generated from the system of model(s). For example, the output can provide feedback for further training, can provide synthetic data for addition to and/or comparison with real captured data, can provide an image processing result, can generate a prediction, etc. Thus, the output 1865 can provide feedback for model training and/or a result of the artificial intelligence task defined by the network of connected models, for example.

At block 1870, the system of model(s) is deployed for further use, execution, etc. For example, the network or system of model(s) that has now been trained/tested to execute one or more artificial intelligence processing tasks can be deployed as a network model, system, or constructed to repeatably execute such tasks in one or more systems. Thus, a user, operator, system, application, etc., can employ the FastEstimator framework to generate a network/system construct to be deployed to execute a defined task or function for its target system, application, etc.

FIG. 19 illustrates a flow diagram of an example method 1900 to configure the example AI model framework 500, 800 to train an AI model and/or execute another AI model task. At block 1910, the pipeline 510 is configured. For example, the data pipeline 510 can preprocess data using one or more preprocessing operations applied to features associated with the data and enable debugging to visualize the preprocessed data, for example. Example preprocessing operations include scaling, clipping, encoding, etc., of the data. As such, one or more preprocessing tasks can be defined and/or otherwise provided to the platform 500, 800, and the framework 500, 800 is then configured to execute those task(s). Each task includes one or more features corresponding to measurable data to be processing using one or more AI models. Preprocessing operations can be applied to the features to set up the AI preprocessing task for execution using the framework 500, 800, for example.

In certain examples, the data pipeline 510 can read cached data files serially or in parallel. Cached data files can include training data, test data, etc. Reading the data files in parallel can improve data read efficiency, for example. In certain examples, the data pipeline 510 can scan hardware information to identify available computing resources and consume the available computing resources for AI modeling training. For example, the data pipeline 510 can determine a number of available processors, etc., and can then use the available processors, etc., to execute the AI task, such as training, testing, etc., an AI model.

At block 1920, the network 820 is configured. For example, the network 820 can instantiate one or more differentiable operations in a training configuration to train an AI model. Differentiable operations can include read/write operations, neural arithmetic units, other parameterized functional blocks, gradient-based optimization, etc. The network 820 can also capture feedback including optimization and loss information to adjust the training configuration. For example, loss information, gradient optimization/improvement information, etc. can be captured as feedback to adjusting the training configuration by adjusting differentiable operations, adjusting weights, adjusting connections, etc. The network 820 can store one or more metrics to evaluate performance of the AI model, for example.

At block 1930, the estimator 830 is configured. For example, the estimator 830 stores the training configuration for the AI model. The estimator 830 configures the pipeline 510 and the network 820 based on the training configuration. The estimator 830 iteratively links the pipeline and the network based on the training configuration and initiates training of the AI model and/or other AI task using the linked pipeline 510 and network 820, for example.

In certain examples, the estimator 830 scans hardware information to identify available computing resources and consume the available computing resources for AI model training and/or other AI task processing. For example, the estimator 830 scans for available processors, etc., and employs the available processors to train the AI model, etc.

At block 1940, one or more AI tasks, such as AI model training, etc., are executed using the configured pipeline 510, network 820, and estimator 830.

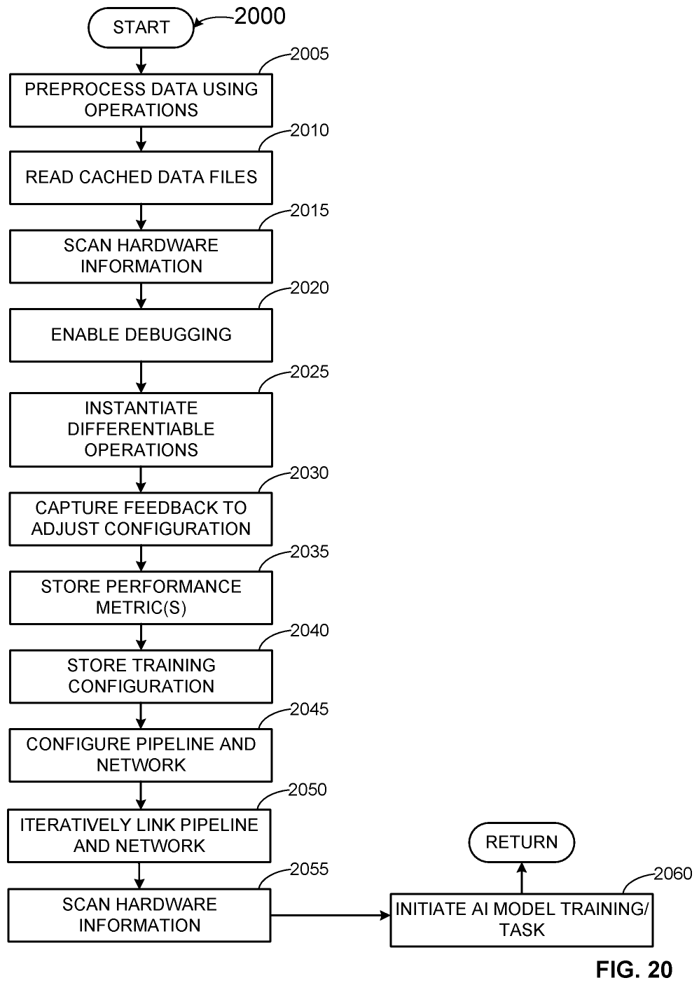

FIG. 20 illustrates a flow diagram of an example method 2000 to configure the example AI model framework 500, 800 to train an AI model and/or execute another AI model task. The example of FIG. 20 is a more detailed example implementation of the method 1900, for example.

At block 2005, the data pipeline 510 preprocesses data using one or more preprocessing operations applied to features associated with the data. Example preprocessing operations include scaling, clipping, encoding, etc., of the data. As such, one or more preprocessing tasks can be defined and/or otherwise provided to the platform 500, 800, and the framework 500, 800 is then configured to execute those task(s). Each task includes one or more features corresponding to measurable data to be processing using one or more AI models. Preprocessing operations can be applied to the features to set up the AI preprocessing task for execution using the framework 500, 800, for example.

At block 2010, the data pipeline 510 reads cached data files in parallel. Cached data files can include training data, test data, etc. Reading the data files in parallel can improve data read efficiency, for example.

At block 2015, the data pipeline 510 scans hardware information to identify available computing resources and consume the available computing resources for AI modeling training. For example, the data pipeline 510 can determine a number of available processors, etc., and can then use the available processors, etc., to execute the AI task, such as training, testing, etc., an AI model.

At block 2020, the data pipeline 510 enables debugging to visualize the preprocessed data. For example, a user, program, interface, system, etc., can visualize the preprocessed data to evaluate the effectiveness of the pipeline 510 and its preprocessing operations in preparing for AI model training, testing, etc.

At block 2025, the network 820 instantiates one or more differentiable operations in a training configuration to train an AI model. Differentiable operations can include read/write operations, neural arithmetic units, other parameterized functional blocks, gradient-based optimization, etc.

At block 2030, the network 820 captures feedback including optimization and loss information to adjust the training configuration. For example, loss information, gradient optimization/improvement information, etc. can be captured as feedback to adjusting the training configuration by adjusting differentiable operations, adjusting weights, adjusting connections, etc.

At block 2035, the network 820 can store one or more metrics to evaluate performance of the AI model. For example, accuracy, precision, processing speed, etc., can be captured and analyzed to evaluate performance of the AI model (e.g., with respect to ground truth, etc.).

At block 2040, the estimator 830 stores the training configuration for the AI model. At block 2045, the estimator 830 configures the pipeline 510 and the network 820 based on the training configuration. At block 2050, the estimator 830 iteratively links the pipeline and the network based on the training configuration. At block 2055, the estimator 830 scans hardware information to identify available computing resources and consume the available computing resources for AI model training and/or other AI task processing. For example, the estimator 830 scans for available processors, etc., and employs the available processors to train the AI model, etc.

At block 2060, the estimator 830 initiates training of the AI model and/or other AI task using the linked pipeline 510 and network 820, for example.

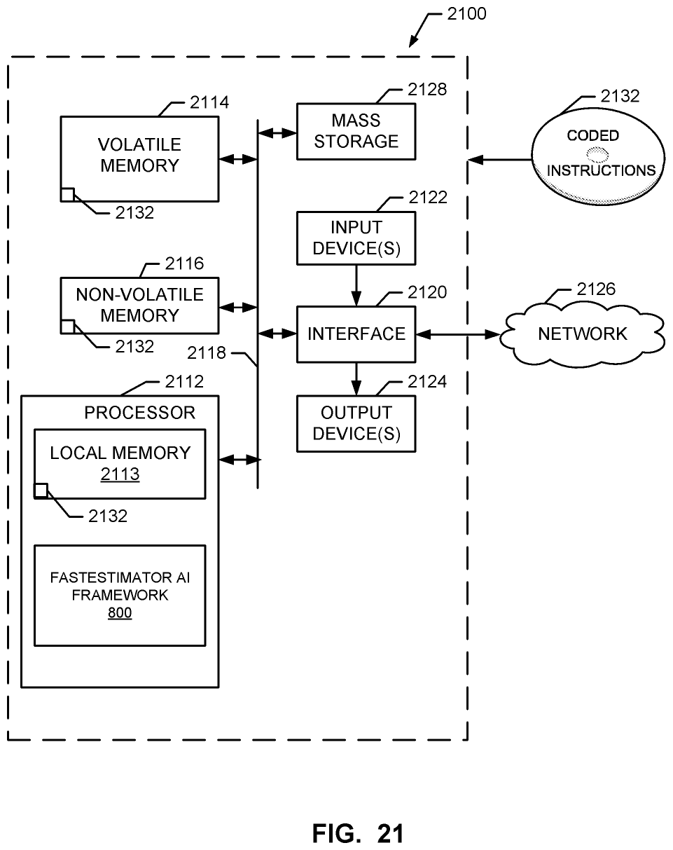

FIG. 21 is a block diagram of an example processor platform 2100 structured to executing instructions to implement the example components disclosed and described herein. The processor platform 2100 can be, for example, a server, a personal computer, a mobile device (e.g., a cell phone, a smart phone, a tablet such as an iPad™), a personal digital assistant (PDA), an Internet appliance, or any other type of computing device.

The processor platform 2100 of the illustrated example includes a processor 2112. The processor 2112 of the illustrated example is hardware. For example, the processor 2112 can be implemented by integrated circuits, logic circuits, microprocessors or controllers from any desired family or manufacturer.

The processor 2112 of the illustrated example includes a local memory 2113 (e.g., a cache). The example processor 2112 of FIG. 21 executes instructions to implement the example FastEstimator AI framework 500, 800, etc. In certain examples, a plurality of processors 2112 and/or processor platforms 2100 can be used to implement individual components of the example FastEstimator AI framework 500, 800 at one or more sites. The processor 2112 of the illustrated example is in communication with a main memory including a volatile memory 2114 and a non-volatile memory 2116 via a bus 2118. The volatile memory 2114 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 2116 may be implemented by flash memory and/or any other desired type of memory device. Access to the main memory 2114, 2116 is controlled by a clock controller.

The processor platform 2100 of the illustrated example also includes an interface circuit 2120. The interface circuit 2120 may be implemented by any type of interface standard, such as an Ethernet interface, a universal serial bus (USB), and/or a PCI express interface.

In the illustrated example, one or more input devices 2122 are connected to the interface circuit 2120. The input device(s) 2122 permit(s) a user to enter data and commands into the processor 2112. The input device(s) can be implemented by, for example, a sensor, a microphone, a camera (still or video), a keyboard, a button, a mouse, a touchscreen, a track-pad, a trackball, isopoint and/or a voice recognition system.

One or more output devices 2124 are also connected to the interface circuit 2120 of the illustrated example. The output devices 2124 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 2120 of the illustrated example, thus, typically includes a graphics driver card, a graphics driver chip or a graphics driver processor.

The interface circuit 2120 of the illustrated example also includes a communication device such as a transmitter, a receiver, a transceiver, a modem and/or network interface card to facilitate exchange of data with external machines (e.g., computing devices of any kind) via a network 2126 (e.g., an Ethernet connection, a digital subscriber line (DSL), a telephone line, coaxial cable, a cellular telephone system, etc.).

The processor platform 2100 of the illustrated example also includes one or more mass storage devices 2128 for storing software and/or data. Examples of such mass storage devices 2128 include floppy disk drives, hard drive disks, compact disk drives, Blu-ray disk drives, RAID systems, and digital versatile disk (DVD) drives.

The coded instructions 2132 representing the instructions executable for the method of FIG. 18 may be stored in the mass storage device 2128, in the volatile memory 2114, in the non-volatile memory 2116, and/or on a removable tangible computer readable storage medium such as a CD or DVD, for example.

From the foregoing, it will be appreciated that the above disclosed methods, apparatus, and articles of manufacture have been disclosed to generate, train, test, and deploy artificial intelligence models using a flexible, definable, reactive platform that provides a framework for model definition, interconnection, training, testing, and deployment. The disclosed methods, apparatus and articles of manufacture improve the efficiency of using a computing device by providing a model-based mechanism to process batch data in connection to an AI computing task to determine, using operators, a network of models and connections to implement a system to execute the AI computing task. The disclosed methods, apparatus and articles of manufacture are accordingly directed to one or more improvement(s) in the functioning of a computer. Further, a new pipelined network estimator architecture is formed to improve the computer's ability to train, test, and deploy robust neural networks and/or other A.I. models.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. An artificial intelligence modularization apparatus comprising:
a data pipeline to:
preprocess data using one or more preprocessing operations applied to features associated with the data; and
enable debugging to visualize the preprocessed data;
a network to:
instantiate one or more differentiable operations in a training configuration to train an artificial intelligence model;
capture feedback including optimization and loss information to adjust the training configuration; and
store one or more metrics to evaluate performance of the artificial intelligence model; and
an estimator to:
store the training configuration for the artificial intelligence model;
configure the pipeline and the network based on the training configuration;
iteratively link the pipeline and the network based on the training configuration; and
initiate training of the artificial intelligence model using the linked pipeline and network.

2. The apparatus of claim 1, further including:
at least one operator, wherein the operator includes a task level computation for data connection to:
link different pre-processing tasks together in the pipeline and route the data to a plurality of locations; and
link one or more differentiable operations together and route the data to a plurality of differentiable operations.

3. The apparatus of claim 1, further including:
at least one trace generator including one or more event functions to be executed in the artificial intelligence model training to:
enable access to the data before and after the artificial intelligence model training for evaluation of the artificial intelligence model training and testing performance;
provide control of training configuration during the artificial intelligence model training; and
communicate data from one event function to another event function within a trace or across plurality of traces for evaluation of the artificial intelligence model training and testing performance.

4. The apparatus of claim 1, wherein the estimator is to iteratively link the data pipeline and the network through a plurality of training loops, each training loop including an epoch loop that includes one or more batch loops.

5. The apparatus of claim 4, wherein the estimator is to generate a trace for each of the plurality of training loops to form a plurality of traces, at least one trace in the plurality of traces to communicate events to a subsequent trace in the plurality of traces.

6. The apparatus of claim 1, wherein the one or more metrics include a metric defined as a value and an update rule to be applied to one or more trainable models.

7. The apparatus of claim 1, wherein the one or more differentiable operations create an expression to construct a graph topology to implement an artificial intelligence task.

8. The apparatus of claim 1, wherein the data pipeline is to read cached data files in parallel to retrieve at least one of training data or testing data.

9. The apparatus of claim 1, wherein at least one of the network or the estimator is to scan for available computing resources and consume the available computing resources to train the artificial intelligence model.

10. At least one computer readable storage medium comprising instructions that, when executed, cause at least one processor to implement at least:
a data pipeline to:
preprocess data using one or more preprocessing operations applied to features associated with the data; and
enable debugging to visualize the preprocessed data;
a network to:
instantiate one or more differentiable operations in a training configuration to train an artificial intelligence model;
capture feedback including optimization and loss information to adjust the training configuration; and
store one or more metrics to evaluate performance of the artificial intelligence model; and
an estimator to:
store the training configuration for the artificial intelligence model;
configure the pipeline and the network based on the training configuration;
iteratively link the pipeline and the network based on the training configuration; and
initiate training of the artificial intelligence model using the linked pipeline and network.

11. The at least one computer readable storage medium of claim 10, wherein the instructions, when executed, cause the at least one processor to:
at least one operator, wherein the operator includes a task level computation for data connection to:
link different pre-processing tasks together in the pipeline and route the data to a plurality of locations; and
link one or more differentiable operations together and route the data to a plurality of differentiable operations.

12. The at least one computer readable storage medium of claim 10, wherein the instructions, when executed, cause the at least one processor to:
at least one trace generator including one or more event functions to be executed in the artificial intelligence model training to:
enable access to the data before and after the artificial intelligence model training for evaluation of the artificial intelligence model training and testing performance;
provide control of training configuration during the artificial intelligence model training; and
communicate data from one event function to another event function within a trace or across plurality of traces for evaluation of the artificial intelligence model training and testing performance.

13. The at least one computer readable storage medium of claim 10, wherein the estimator is to iteratively link the data pipeline and the network through a plurality of training loops, each training loop including an epoch loop that includes one or more batch loops.

14. The at least one computer readable storage medium of claim 13, wherein the estimator is to generate a trace for each of the plurality of training loops to form a plurality of traces, at least one trace in the plurality of traces to communicate events to a subsequent trace in the plurality of traces.

15. The at least one computer readable storage medium of claim 10, wherein the data pipeline is to read cached data files in parallel to retrieve at least one of training data or testing data.

16. The at least one computer readable storage medium of claim 10, wherein at least one of the network or the estimator is to scan for available computing resources and consume the available computing resources to train the artificial intelligence model.

17. A computer-implemented method comprising:
preprocessing, with a data pipeline, data using one or more preprocessing operations applied to features associated with the data;
enabling, with the data pipeline, debugging to visualize the preprocessed data;
instantiating, with a network one or more differentiable operations in a training configuration to train an artificial intelligence model;
capturing, with the network, feedback including optimization and loss information to adjust the training configuration;
storing, with the network one or more metrics to evaluate performance of the artificial intelligence model;
storing, with an estimator, the training configuration for the artificial intelligence model;
configuring, with the estimator, the pipeline and the network based on the training configuration;
iteratively linking, with the estimator, the pipeline and the network based on the training configuration; and
initiating, with the estimator, training of the artificial intelligence model using the linked pipeline and network.

18. The method of claim 17, further including:
linking pre-processing tasks in the pipeline and routing the data to a plurality of locations using an operator; and
linking one or more differentiable operations and routing the data to a plurality of differentiable operations using the operator.

19. The method of claim 17, further including:
enabling access to the data before and after the artificial intelligence model training for evaluation of the artificial intelligence model training and testing performance;
providing control of training configuration during the artificial intelligence model training; and
communicating data from one event function to another event function within a trace or across plurality of traces for evaluation of the artificial intelligence model training and testing performance.

20. The method of claim 10, wherein the estimator is to iteratively link the data pipeline and the network through a plurality of training loops, each training loop including an epoch loop that includes one or more batch loops.
